(19) United States
(12) Patent Application Publication
(10) Pub. No.: US 2018/0144814 A1
(43) Pub. Date: May 24, 2018

(54) Systems and Methods for Facilitating Coding of a Patient Encounter Record Based on a Healthcare Practitioner Recording

(71) Applicant: Technology and Innovation Fund, LP, St. Helier (JE)

(72) Inventor: John Thomas Bright, Georgetown, TX (US)

(73) Assignee: Technology and Innovation Fund, LP, St. Helier (JE)

(21) Appl. No.: 15/787,688

(22) Filed: Oct. 18, 2017

Related U.S. Application Data
(60) Provisional application No. 62/410,055, filed on Oct. 19, 2016.

(51) Int. Cl. G16H 10/60; G16H 40/63; G06N 5/02

(52) U.S. Cl. CPC G16H 10/60; H04L 63/0428; G06N 5/02; G16H 40/63

(57) ABSTRACT

Systems and methods for patient encounter data capture, coding and billing are provided. Systems and methods are disclosed which may include one or more of voice enabled live stream, recording and messaging capabilities, along with a telemedicine interface and workflow that enable clinical documentation improvement coders (CDICs) the ability to document and code a medical encounter, flag the record for physician/provider review and sign-off, and filing a claim with the insurance provider in a secure manner.

### CROSS REFERENCE TO RELATED APPLICATIONS

This application claims the benefit of the filing date of U.S. provisional patent application number Ser. No. 62/410,055, titled ‘Systems and Methods for Facilitating Coding of a Patient Encounter Record Based on a Healthcare Practitioner Recording’, which was filed in the USPTO on Oct. 19, 2016 and which includes the same inventor. That provisional application is hereby incorporated by reference as if fully set forth herein.

### FIELD OF THE TECHNOLOGY

Patient encounter capture, coding and medical billing systems and methods are disclosed. Systems and methods are disclosed which may include one or more of voice enabled live stream, recording and messaging capabilities, along with a telemedicine interface and workflow that enable clinical documentation improvement coders (CDICs) the ability to document and code a medical encounter, flag the record for physician/provider review and sign-off and filing a claim with the insurance provider in a secure manner that is complies with the Health Insurance Portability and Accountability Act of 1996 (HIPAA) and the final omnibus rule that implements a number of provisions of the HITECH Act.

### BACKGROUND OF THE TECHNOLOGY

Physicians in the U.S. are required to perform administrative tasks, which impede their ability to provide patient related services. The more time they spend with administrative issues, the less time they have for patients. These administrative tasks, among other things, require documentation of specific medical record data sets for every doctor/patient interaction under the CMS Medicare/Medicaid Guidelines for Evaluation and Management (E&M). Additionally, physicians are often required to follow specific structured guideline formats for the timing and content of these data sets or they risk reimbursement issues.

Some physicians have employed scribes to shadow them on patient encounters and document each encounter. However, the scribes do not perform the ICD-10 coding. Further, since most physicians do not have the training to properly code encounter records for medical billing they often need to also employ coding specialists. Additionally, many physicians must also hire a billing admin who will prepare and provide the bill to the insurance company. Thus, for each patient encounter the physician has hired up to 3 additional people just to service the administrative tasks.

In view of these deficiencies in traditional physician operations, the instant disclosure identifies and addresses a need for improved systems and methods for patient encounter data capture, coding and billing.

### BRIEF SUMMARY OF THE TECHNOLOGY

Many advantages of the technology will be determined and are attained by the technology, which in a broad sense provides systems and methods for patient encounter data capture, coding and billing.

In one or more implementations of the technology, a method is provided for coding an encounter between a patient and a healthcare practitioner. The method may include capturing the encounter with a device associated with the healthcare practitioner and transmitting the captured encounter over a network to a remote server for storage.

The method may also include transmitting the captured encounter for review by a clinical documentation improvement coder (CDIC) and receiving input from the CDIC. The input from the CDIC may include a patient encounter record which includes at least one code entry associated with the captured encounter. The method may also include transmitting the patient encounter record over the network to a remote electronic medical record (EMR) system for storage.

In one or more implementations of the technology, a system is provided for coding an encounter between a patient and a healthcare practitioner. The system may include a processor based device that includes a non-transitory computer readable medium that stores instructions which when executed cause the device to capture the encounter. The instructions may also cause the device to transmit the captured encounter over a network to a remote server for storage and receive input from a remote clinical documentation improvement coder (CDIC). The input from the CDIC may include a patient encounter record which includes at least one code entry associated with the captured encounter. The instructions may cause the device to receive input regarding the patient encounter record from the healthcare practitioner and transmit the patient encounter record with the healthcare provider input over the network to a remote electronic medical record (EMR) system for storage.

Features from any of the above-mentioned embodiments and/or examples may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

For a better understanding of the technology, reference is made to the following description, taken in conjunction with the accompanying drawings, in which like reference characters refer to like parts throughout, and in which:

FIG. 1 is a block diagram illustrating a system in accordance with one or more embodiments of the technology;

FIG. 2 is a flowchart illustrating an aspect of the system of FIG. 1 in accordance with one or more embodiments of the technology;

FIG. 3 is another flowchart illustrating an aspect of the system of FIG. 1 in accordance with one or more embodiments of the technology;

FIG. 3a is a flowchart illustrating another aspect of the system of FIG. 1 in accordance with one or more embodiments of the technology;

FIG. 4 is yet another flowchart illustrating an aspect of the system of FIG. 1 in accordance with one or more embodiments of the technology;

FIG. 5 is still another flowchart illustrating an aspect of the system of FIG. 1 in accordance with one or more embodiments of the technology;

FIG. 5a is a flowchart illustrating another aspect of the system of FIG. 1 in accordance with one or more embodiments of the technology;

FIG. 6 is another flowchart illustrating an aspect of the system of FIG. 1 in accordance with one or more embodiments of the technology;

FIG. 7 illustrates a block diagram providing elements present in the devices in the system of FIG. 1.

The technology will next be described in connection with certain illustrated embodiments and practices. However, it will be clear to those skilled in the art that various modifications, additions, and subtractions can be made without departing from the spirit or scope of the claims.

### DETAILED DESCRIPTION OF THE INVENTION

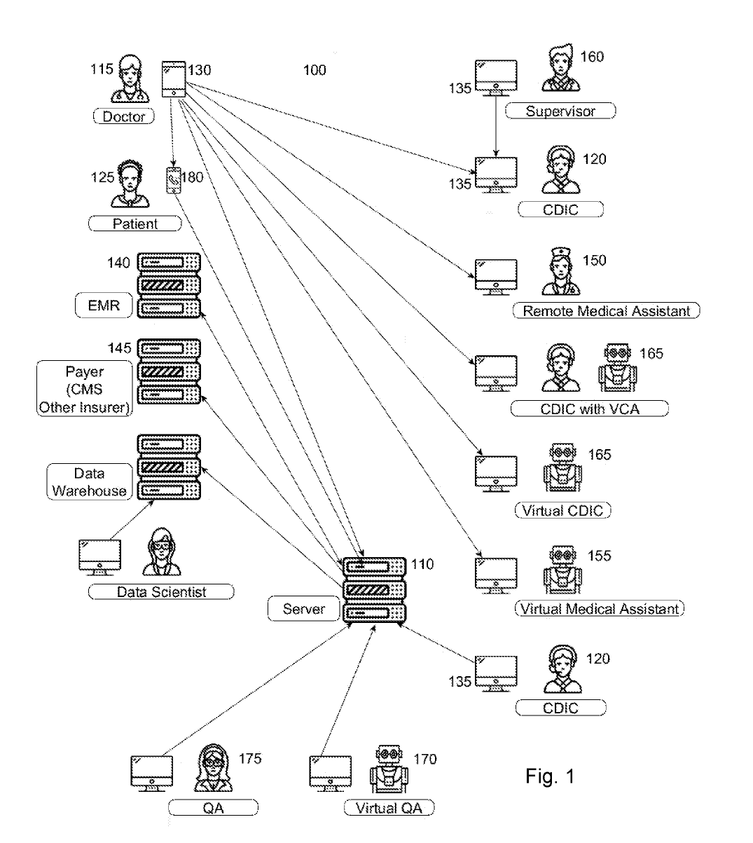

Referring to the drawings in detail wherein like reference numerals identify like elements throughout the various figures, there is illustrated in FIGS. 1-7 systems and methods for patient encounter data capture, coding and billing. Principles and operations of the technology may be better understood with reference to the drawings and the accompanying description.

Discussion of an embodiment, one or more embodiments, an aspect, one or more aspects, a feature, one or more features, or a configuration or one or more configurations, an instance or one or more instances is intended be inclusive of both the singular and the plural depending upon which provides the broadest scope without running afoul of the existing art and any such statement is in no way intended to be limiting in nature. Technology described in relation to one or more of these terms is not necessarily limited to use in that embodiment, aspect, feature or configuration and may be employed with other embodiments, aspects, features and/or configurations where appropriate.

For purposes of this disclosure “computing platform”, “computer”, or “device” means a mobile phone, laptop computer, tablet computer, personal digital assistant (“PDA”), desktop computer, electronic reader (“e-reader”), mobile game console, smart watch, smart glasses, voice assistant devices, or any other mobile device which can run software applications (“apps”) and transmit and receive data. It may also be used to refer to peripheral devices used with such devices (e.g. cameras, microphones, speakers, displays, etc.)

For purposes of this disclosure “remote” means accessible via a network, telephone, email, text, video, website or a combination of the same or any other form of communication wherein the parties need not be collocated to communicate.

For purposes of this disclosure “app” means a software application that can be run on a computing platform which has an operating system (e.g. Windows™, iOS™, Android™, etc.). It may also include a web accessible application.

For purposes of this disclosure “Cloud”, “Web”, and/or “Internet” shall be used interchangeably herein and shall refer to the global wide area network referred to as the world wide web.

For purposes of this disclosure a “CDIC” is a skilled person who is capable of properly documenting patient encounters in an electronic medical record (EMR) system and who is able to properly code a patient encounter using ICD-9, ICD-10 and/or any other required coding system. A CDIC is capable of understanding medical terminology from general and/or specific medical practice disciplines. Like doctors, a CDIC may be specialized for a particular area of medicine or be a generalist. CDICs may be organized into teams and pre-allocated to work encounters for one or more clients (Primary Healthcare Organizations/Providers); a planning process known as “Work Scoping” and “Work Mapping”. Work may be shared amongst the team on a first come first served basis to ensure minimal completion times and maximum utilization of resources or one or more CDICs may be assigned to a specific client, task and/or medical specialty.

For purposes of this disclosure a “healthcare provider”, “healthcare professional”, “physician” and “healthcare practitioner” means a person, group of people, hospital, or other business concern that provides health related services to a patient such that payment for the services may qualify for health insurance reimbursement.

For purposes of this disclosure a “DVR” means an audio, video or audio/video recording that can be navigated even if the real-time recording is still taking place.

Referring to FIG. 1, technology developed for a patient encounter clinical system 100 includes hardware and software. A service provider has one or more servers 110 for secure data storage and authentication of users of the system 100. The secure data storage server 110 may be configured with one or more applications which store digital voice recordings, photos, videos and/or data, using a secure connection and storage. The recordings, data and other information may be stored in a folder structure and database system or in some other suitable storage format. The service provider server 110 enables a physician 115 to stream live audio to the server 110 where it may be stored. In one or more embodiments, the physician may stream live audio and video, audio only, or video only to the server 110 for recording. Additionally, or alternatively, the physician 115 may record one or more of audio only, video only or audio/video to a local device 130 and upload the recorded patient encounter at a later time. Any or all of the above recordings may be DVR recordings. The physician may also be provided with the option for real-time secure messaging with a CDIC 120 using a secure connection. The patient encounter can be in person or a remote encounter (e.g. telemedicine). During the encounter, the physician 115 can request labs or other tests for a patient 125 from the CDIC 120, a remote medical assistant 150 or an artificial intelligence during the encounter using the physician application. The physician application may also be configured to provide direct access for the physician to order such tests or extras directly (e.g. contact pharmacy, testing lab, etc.). The application may employ voice recognition for such operations and/or on screen navigation with an input device such as a mouse or keyboard. The physician may be prompted to follow an encounter guide to streamline the patient encounter and ensure all the steps are completed to facilitate accurate and complete encounter coding.

The physician 115 may be authenticated through an authentication server (not shown) then provided access to the service provider server 110 applications using a token system ensuring only an authenticated physician 115 can access the service provider server 110 while all other traffic is prevented access to confidential server resources. Other non-token based secure authentication based systems may be employed without departing from a scope of one or more claims. The service provider server 110 may connect to the Cloud through a secure connection using Secure Socket Layer (SSL) allowing a device and/or the CDIC file manager workstations 120 to connect to applications and resources located on service provider server 110.

Physician 115 may use a device 130 configured for the system. The device 130 may connect to the service provider server 110 through an app located on the device 130 using a secure connection and require authentication from the authentication server. The physician provider may be required to login to the app and may be required to enter various credentials for the first login and/or for subsequent logins. Physician 115 may utilize the device 130 to enable live audio streaming and DVR recording while conducting a patient encounter. The app may access a camera and microphone associated with device 130 for this feature. One or more of these may be integral with device 130 or adjunct thereto. Physicians may record audio, video and/or pictures and upload the recorded content to the service provider server 110 and request other services (e.g. lab services, other test, consults, etc.) to be performed by a CDIC 120 that is documenting the patient encounter (or by some other entity that may be available through the physician application). Physicians may communicate in real-time using the secure messaging application provided on the device 130. The message application may provide the ability to communicate using text, video, and/or voice and may be included in the physician app or a separate app. Device 130 may support the health insurance portability and accountability act (HIPAA) security rules and requirements.

A CDIC 120 may document and code the encounter using a CDIC file manager workstation 135. The CDIC file manager workstation 300 may include an application that provides access to DVR recordings and/or photos uploaded by the physician 115. The CDIC 120 may connect to an EMR system 140 that the physician utilizes to document patient encounters and create a patient encounter by entering in findings provided by the captured DVR. The CDIC 120 codes each encounter in preparation of billing the patient's insurance company 145. The CDIC file manager workstation 135 enables a CDIC 120 to send and receive secure messages with the physician 115 in real-time to ensure a high level of communication.

The following description provides various exemplary embodiments of the technology. Physicians 115 utilize the physician app, which is installed on a device 130. The app enables the physician 115 to capture an audio recording of a patient encounter, and securely stream (SSL) the recording in real-time, or optionally, at a later date, to server 110 for secure storage and playback by CDICs 120 through the CDIC workstation 135.

The physician app may present a physician with an up to date list of patient appointments throughout the day, week, month (or whatever granularity is designed—and may provide a choice to switch between granularities), via a real-time electronic link established between the provider's EMR/Booking systems 140 and the server 110, based on the Health Level Seven International (HL7) standards for the exchange, integration, sharing, and retrieval of electronic health information. It is possible that the required security standard may change in which case the security level of the link would change to meet the required level.

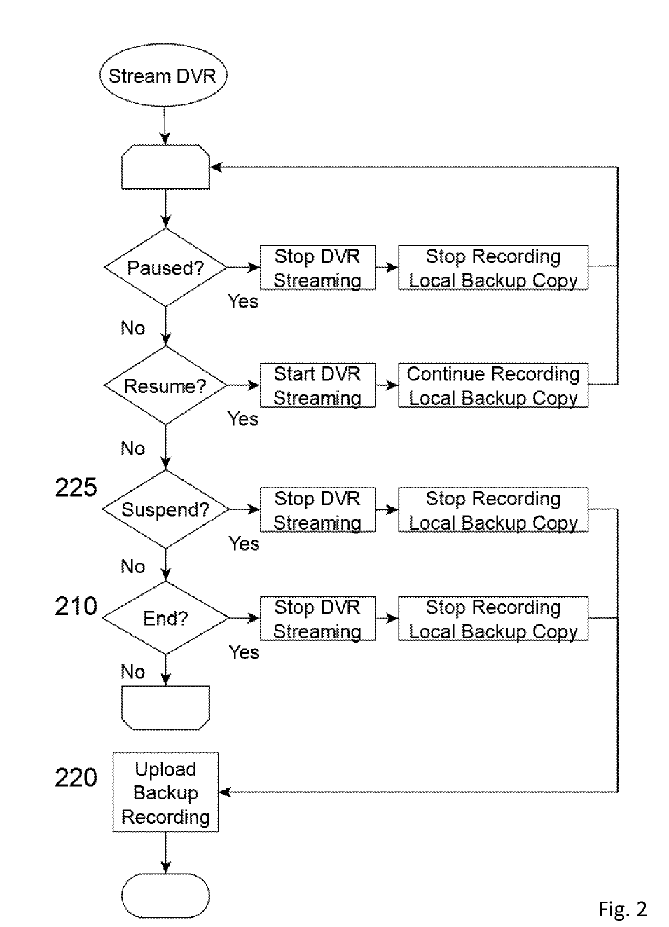

As illustrated in FIG. 2, the physician selects the current appointment from the presented list and initiates DVR recording 200 at the beginning of the patient encounter. The recording may begin slightly before the encounter or slightly after the start and still fall within a scope of one or more claims. Throughout the encounter, the physician may pause and resume DVR streaming as necessary, mark the encounter as STAT (urgent) for the attention of the next available CDIC 120, and/or capture pictures and notes via the physician app as additional supporting information for CDICs 120 when coding the patient encounter. If the encounter is being stored locally, then at the end of the encounter (or slightly before or after) the physician stops the DVR stream 210 and the physician app (automatically or at the request of the physician) securely uploads any supporting pictures and notes captured throughout the encounter and marks the DVR recording on the server as “Submitted” 220. If the physician is called away during the encounter 215, the physician may suspend the encounter in the physician app 225, in which case the DVR recording stops and may be uploaded to the server 110 in which case the DVR recording on the server 110 is marked as “Suspended”. In the event that the encounter is being live streamed to the server 110 then the pictures, notes etc. may be uploaded at the end of the encounter or during the encounter depending upon the design of the system.

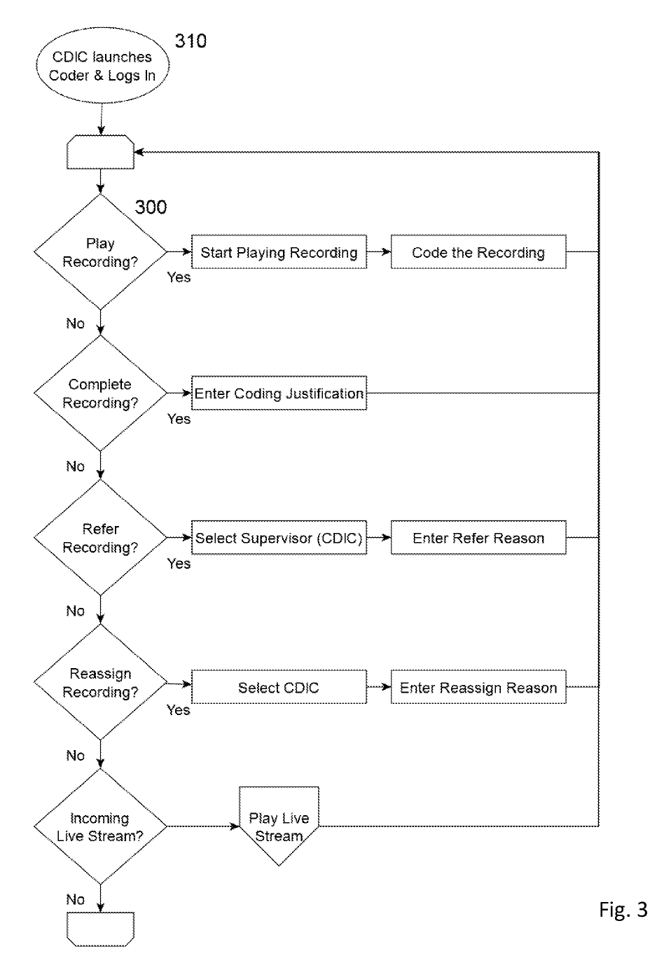

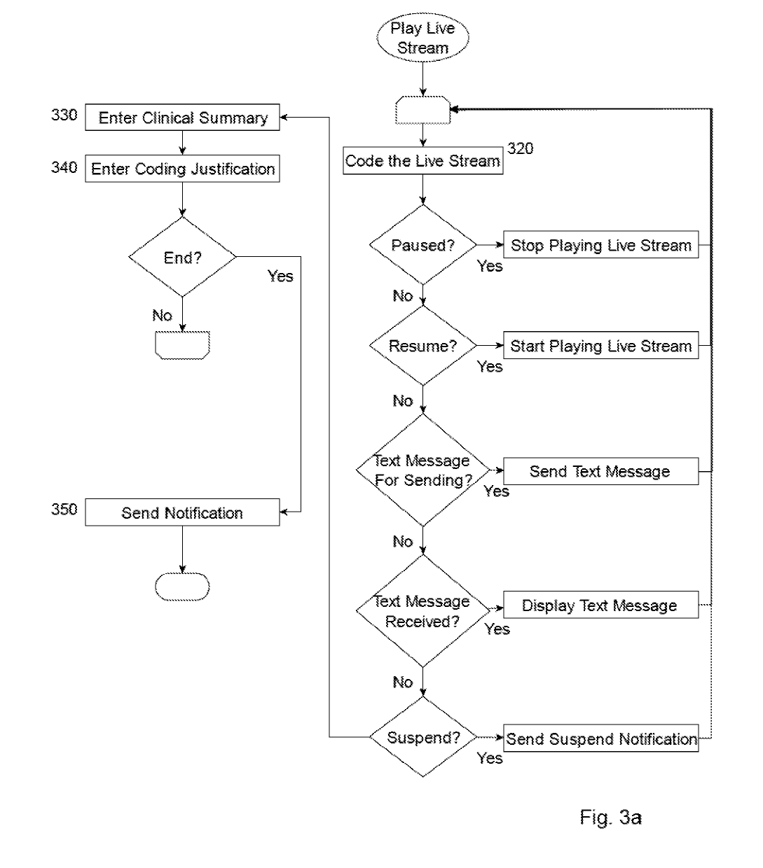

As illustrated in FIG. 3, a CDIC 120 may utilize workstation 135, to view, grab (assign and lock recording) and playback “In-Progress” and/or “Submitted” recordings.

The workstation 135 may include a fully featured media player allowing quick and easy navigation of a recording (e.g. pause, play, go to start, go to end, fast forward, reverse, skip forward, skip back), with the ability to add bookmarks (with labels) as the recording is being streamed/played (for revisiting sections of the audio recording later).

A CDIC will typically use a Remote Desktop Protocol (RDP) client deployed on their computer to securely login to the Provider's EMR system 310 and ICD-9 and ICD-10 code (or some other standard code) a patient encounter 320. The CDIC 120 may enter a clinical summary of the encounter 330 on the patient's records for subsequent distribution to the patient.

The workstation media player may be keyboard and/or voice enabled and compatible with a set of peripheral devices (e.g. foot pedals) to enhance productivity, allowing the CDIC 120 to maintain focus on entering the appropriate codes and clinical summary into a patient's record (within the provider's EMR system) while navigating (forward and/or back) through the recording.

Upon completing the coding of a patient encounter, a CDIC 120 may enter a coding justification 340, along with the codes used and a copy of the clinical summary, into in the workstation app. The physician 115 may then be notified 350 via the physician app that the coding has been completed. The physician 115 may then review and/or approve the updates to the patient's record within the EMR system, or make amendments (if applicable and if allowed by the facility) to the CDIC's coding of the encounter 360. The coding justification may be viewable through the physician app and the physician 115 may be required to electronically sign their approval within the app, or enter their reasons for amending the codes used by the CDIC 120. A record may be retained of all changes made to the coding.

From the perspective of the Centers for Medicare and Medicaid Services (CMS), and the Medicare Administrative Contractors (MAC) that operate under the jurisdiction of the CMS, and Private Healthcare Insurance Operators, the process described above provides a significant and highly effective deterrent to physicians against the fraudulent practice of over-coding patient encounters to submit false claims and receive overpayments.

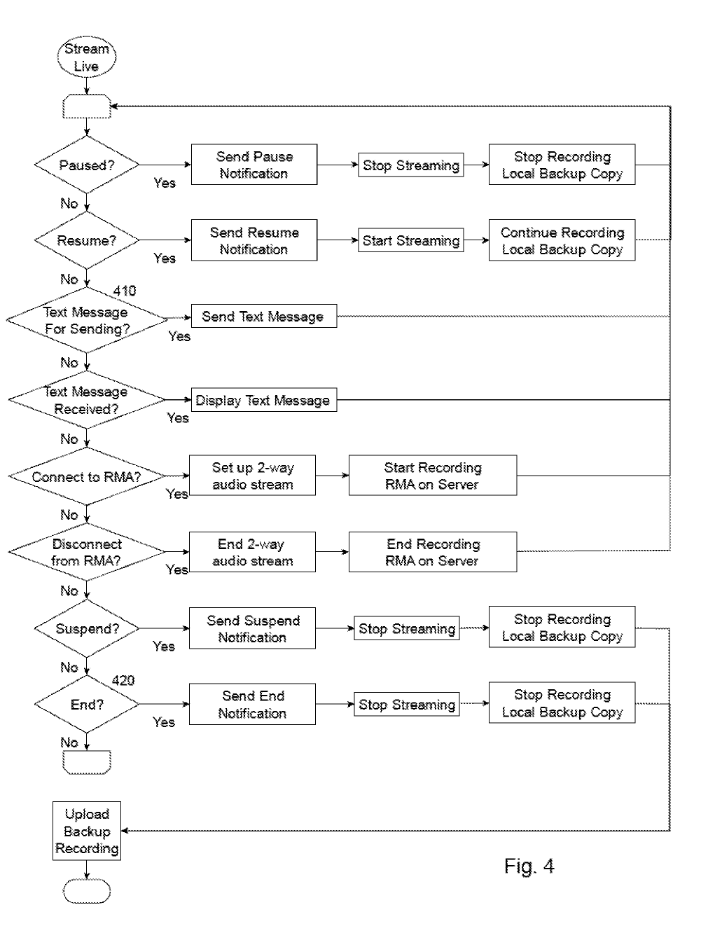

In one or more embodiments, FIG. 4, the physician 115 may be connected to an available CDIC 120 at the beginning of the patient encounter, so the CDIC can listen to a live stream and code the patient encounter as it is taking place 400. Through the physician app, the physician 115 may request a live (real-time) connection to an available CDIC 120 and interact with the CDIC 120 through secure 1 and/or 2-way voice and text messaging 410. All communications between the physician 115 and CDIC 120 may be streamed to the server 110 and recorded along with the patient encounter for secure storage and SSL playback/review (e.g. Quality Assurance & Training purposes).

The workstation 135 may include a fully featured voice and/or video conferencing capability that enables the CDIC 120 to accept a live connection request and communicate with the physician 115 in real-time as the CDIC 120 listens to and codes the patient encounter. At the end of the patient encounter, the physician 115 may communicate any closing remarks/instruction to the CDIC 120, 420. The CDIC 120 may complete final (coding & clinical summary) changes to the patient's record within the provider's EMR system 140 and enter the coding justification into the workstation 135 for the physician to review and approve or amend 430. This provides the ability to have the patient encounter fully coded and completed before the beginning of the next patient appointment.

The CMS stipulates, in its literature, that giving a clinical summary (defined as “after visit summary” (AVS)) to a patient after each office visit is an element of a meaningful use of an electronic health record (EHR) stage one. The disclosed technology provides a physician with the ability to provide patients with a copy of their AVS at the end of their visit and assists providers with meeting their Meaningful Use obligations.

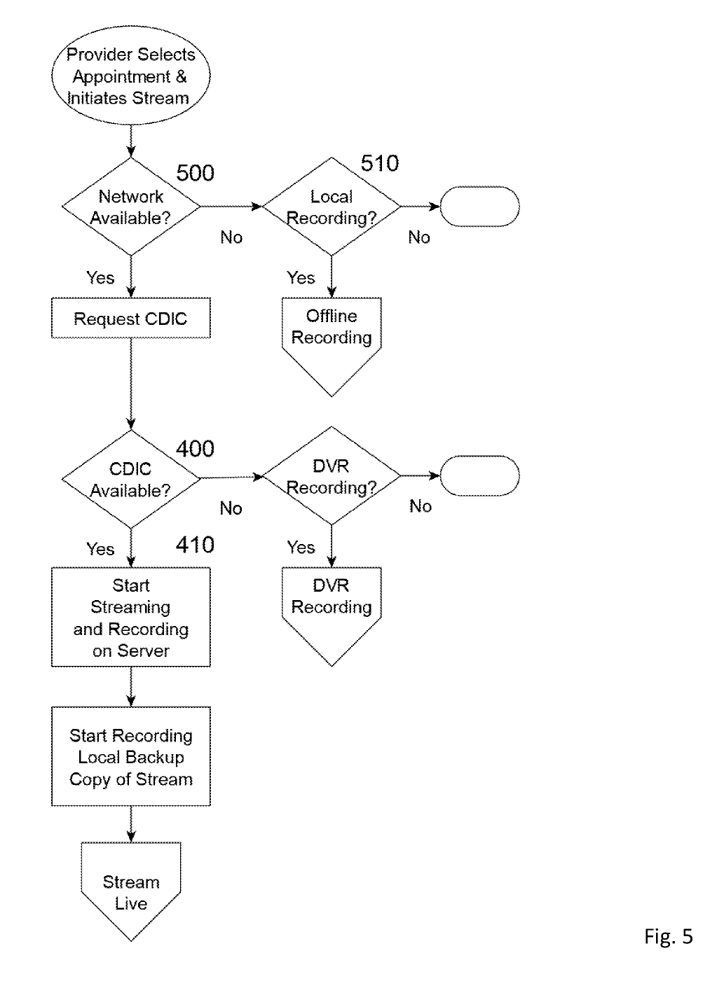

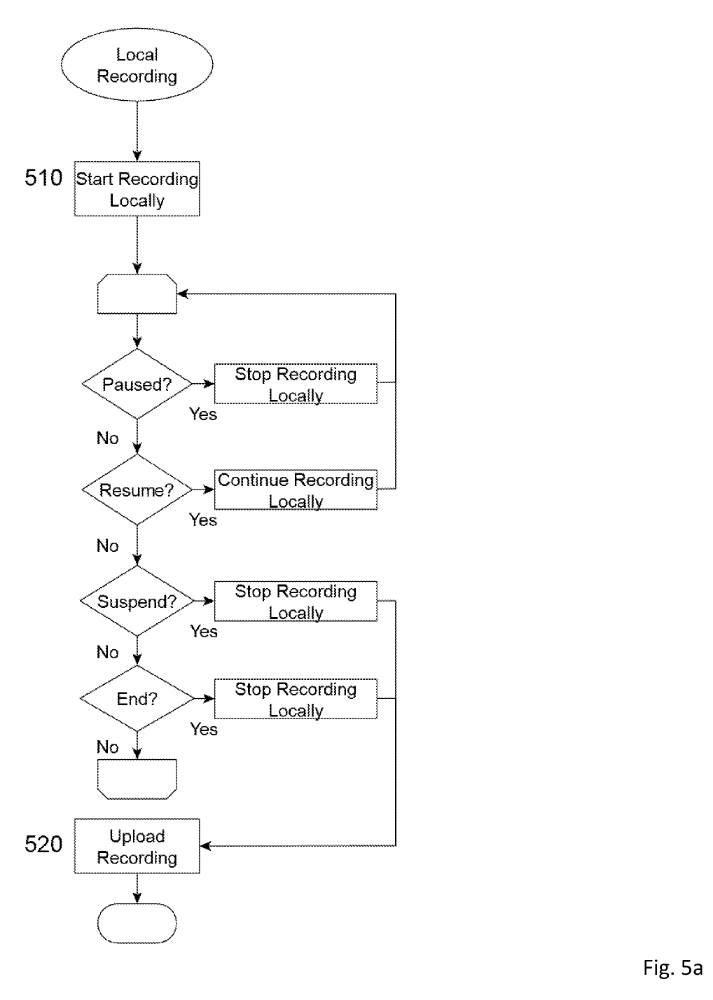

In one or more embodiments, FIG. 5, the physician app may switch (automatically or manually) to offline recording mode if the connection to the server 110 cannot be maintained (e.g. due to network problems, data center problems, server downtime, etc.). Upon detecting a loss of connection, the app may attempt to reconnect to the server 110. If the connection becomes unstable or drops completely, the app may automatically switch to offline recording mode or provide an indication to the physician 115 that the system should be switched to offline mode 500, thus allowing the physician 110 to continue to record the patient encounter while securely storing the recording on the local device. The stored encounter may then be automatically or manually submitted for coding when the connection is returned and/or at the end of the encounter 520.

This allows physicians 115 to continue to operate even where DVR or live streaming is transmitted over unreliable networks and/or network coverage cannot be guaranteed. The physician app may also allow physicians 115 to record and submit additional instructions (Addendums) for a CDIC 120 after the initial encounter has been completed. These Addendums may be written and/or audio case notes. Addendums may separate files which may be linked to the original DVR. The CDIC 120 may access the Addendums via the workstation 135 in the same way as the encounter recordings are accessed.

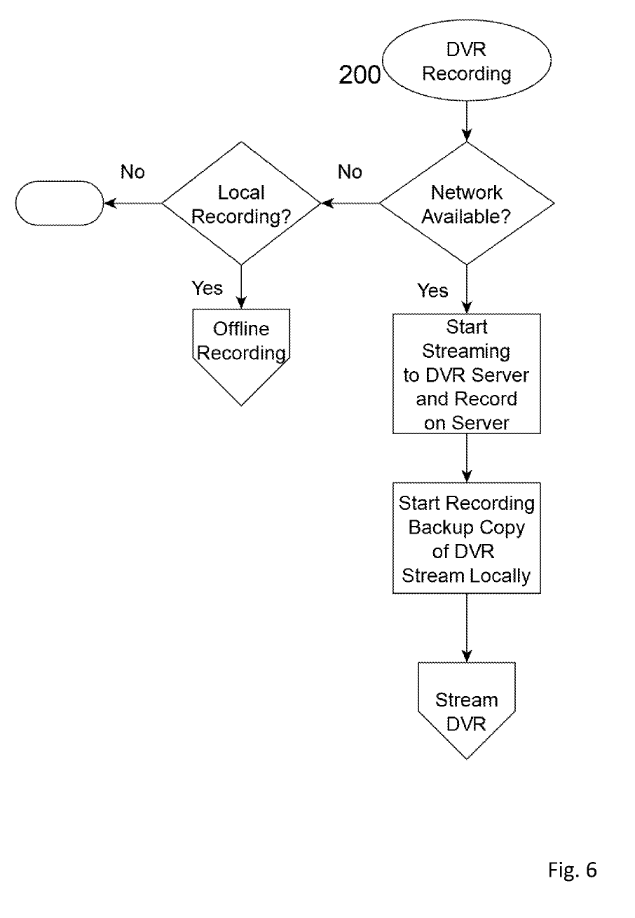

In one or more embodiments, FIG. 6, the disclosed technology supports the use of remote medically trained and qualified human resources (e.g. nurses, specialist medical practitioners and other doctors) 150 to assist physicians 115 during a patient encounter. In much the same way that a secure connection is established between the physician 115 and a CDIC 120 through the physician app, the physician 115 may be provided with an option 600 to connect to a remote medical assistant (RMA) 150. The connection may provide voice, text and/or video capabilities between the physician 115 and the RMA 150. The CDIC 120 may be included in the physician/RMA connection or excluded. The RMA may also be provided with a remote desktop protocol (RDP) or some other secure access to the provider's EMR system 140, 610. Communications (voice, video and text conversations) between the physician 115 and an RMA 150 may be streamed to the server 110 and recorded along with the patient encounter 620.

In one or more embodiments, the disclosed technology may provide artificial intelligence (AI) accessed through the physician app which acts as a virtual medical assistant (VMA) 155. The AI may be employed manually and/or it may be accessed via voice recognition technology. When integrated with the provider's EMR system 140, the AI may perform medical assistant tasks (e.g. check for contraindications against the patient's medical history when prescribing a new drug). With access to the patient's medical history, a VMA 155 may prompt the physician 115 at the start and throughout the encounter with relevant information, advice and medications suitable for the patient's current condition. Machine learning (both supervised and unsupervised), rules-based expert systems (trained by medical experts across multiple fields) and other AI techniques may be used to train and continually improve the VMAs 155.

In one or more embodiments, the disclosed technology may enable the defining, populating and updating of profiles for the CDICs 120, RMAs 150 and physicians 115. Profiles may be employed to connect physicians 115 with appropriate CDICs 120 and/or RMAs 150 (e.g. CDICs who are proficient with a client's EMR system, common RMA and physician medical specialties, client location and RMA locations and time zones). The workstation 135 may be employed to manually match physicians 115 with CDICs 120/RMAs 150 and/or to define rules for automated matching. Matching can be performed via Boolean or natural language searching and/or by rules based decision models using profile information and past performance measurements for automated matching purposes.

In one or more embodiments, the disclosed technology may enable resource balancing for the CDIC 120 and RMA 150 service providers. Reporting dashboards within the workstation 135 may provide a real-time view of workloads across an organization compared to client service level agreements (SLAs). Where necessary, resources (teams and individuals) may be manually or automatically reassigned to balance workloads and ensure that SLAs are met. Resource balancing rules and models may be employed for automated rebalancing along with adaptive control techniques (based on work performance measurement and outcome data captured by the system) to improve the accuracy of rules and models over time.

In one or more embodiments, the disclosed technology may enable live coding support for CDICs 120. A CDIC 120 may request real-time input from their supervisors 160 and peers as they are coding an encounter, by initiating a voice or video conference through the workstation 135. One or more additional participants can then follow the encounter recording simultaneously with the CDIC 120 and provide the necessary input and direction required to properly code the encounter. For live stream encounters, the additional participants may be connected into the live stream such that they may communicate with CDIC 120 (and Physician 115, if necessary) through both text messaging and 2-way voice and video. In one or more embodiments, the additional participants may only be provided access to the original CDIC 120. Whether the additional participant(s) can communicate with the physician 115 or not, all communications between participants may be streamed to the server 110 and recorded along with the patient encounter for secure storage and playback/review.

In one or more embodiments, the disclosed technology may provide AI support which acts as a virtual coding assistant (VCA) 165. The VCA 165 may assist the CDIC 120 in identifying appropriate codes to be used for an encounter. The AI may be employed manually and/or it may be accessed via voice recognition technology. It may provide a pre-filled patient encounter report based on keywords and it may provide navigational support for the CDIC 120 by allowing the CDIC 120 to skip to bookmarks and/or to skip certain portions of the recorded encounter (e.g. small talk). Machine learning (both supervised and unsupervised), rules-based expert systems (trained by CDI and medical experts across multiple fields) and other AI techniques may be employed to train and continually improve VCAs 165. The VCA's 165 may be configured to automatically populate a coding field and/or to provide a list of suggestions to the CDIC 120.

In one or more embodiments, the VCA 165, may produce a real-time transcription of the patient encounter, selection of the appropriate codes for the encounter and generation of the clinical summary and coding justification. The results would be reviewed by the CDIC 120 for accuracy and completeness and amended as necessary. Amendments by CDICs 120 may be captured by the system and used with quality performance measurement and outcome data to train VCA models to become more accurate over time and possible replace the CDIC 120 altogether.

In one or more embodiments, the VCAs 165 may provide real-time or subsequent language translations of patient encounters. For example, if the patient and physician are conducting the encounter in Spanish, the encounter may still need to be officially recorded in English when submitted to the EMR system 140. Furthermore, two versions of the clinical summary may be required, one in Spanish for the patient and one in English as the official record of the encounter.

In one or more embodiments virtual quality assurance (VQA) AI 170, similar to VCAs 165, may be used to review a random or not random selection of completed encounters and flag suspect cases for human review (HQA) 175. Results from VQA 170 and HQA 175 checks may be securely stored in the server 110 and used to train staff and AI models underpinning VCAs and VQAs.

In one or more embodiments a patient 125 may be provided an option to sign up for receipt of their clinical summaries via a secure patient portal which would reside on a patient device 180 or on a server accessible by an app located on a patient device. The patient portal may also provide patients 125 with (current and historical) details of their conditions, medications and appointments, general healthcare advice (relevant to their condition and medications) and scheduling and tracking services (e.g. book and cancel appointments, track and request prescriptions). The use of AI technologies within the patient portal may enable context based information to be served to the patient 125.

The patient portal may be used to deliver telemedicine services, allowing the patient to request and initiate/participate in a telemedicine encounter through a voice and/or video link with their physician 115, who in turn may initiate a telemedicine encounter via the physician app with the CDIC 120. The telemedicine encounter may be either relayed (live stream) to the CDIC 120 or recorded (DVR stream) for the CDIC 120 to playback when they are available.

In one or more embodiments coding data stored within the server 110 may be securely submitted to the CMS 145 and other insurance payers and used to validate the accuracy (or otherwise) of equivalent data submitted by primary healthcare providers via their EMR systems 140.

In one or more embodiments data from multiple servers 115 may be aggregated along with data contributions from insurance payers (e.g. CMS and primary healthcare institutions) to create predictive models that can be deployed with VCAs and VQAs to spot potential fraud (e.g. over-coding) prior to an insurance claim being submitted.

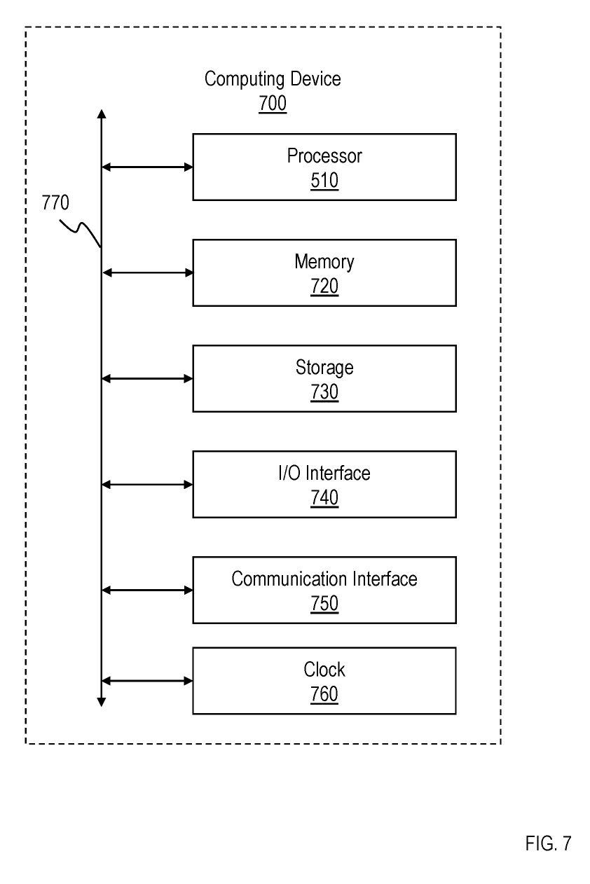

FIG. 7 very generally illustrates elements that will be present in each of the devices illustrated in FIG. 1. These are conventional elements and thus their operation and interconnections will not be further discussed herein. Those of ordinary skill in the art are deemed to understand how elements such as processor 710 memory 720, storage 730, input/output (“I/O”) interface 740, communications interface 750 and clock 760 send and/or receive messages via bus 770. While these elements are not illustrated in the block diagram of FIG. 1, those skilled in the art will recognize that the various devices 110, 130, 135, 140, 145, 180 each includes, among others, these elements and that the interaction between 2 or more of these elements is employed to perform the functions of the disclosed technology.

Having thus described preferred embodiments of the technology, advantages can be appreciated. Variations from the described embodiments exist without departing from the scope of the claims. Thus it is seen that systems and methods for patient encounter data capture, coding and billing are provided. Although embodiments have been disclosed herein in detail, this has been done for purposes of illustration only, and is not intended to be limiting with respect to the scope of the claims, which follow. It is contemplated by the inventors that various substitutions, alterations, and modifications may be made without departing from the spirit and scope of the technology as defined by the claims. Other aspects, advantages, and modifications are considered within the scope of the following claims. The claims presented are representative of the technology disclosed herein. Other, unclaimed technology is also contemplated. The inventors reserve the right to pursue such technology in later claims.

Insofar as embodiments of the technology described above are implemented, at least in part, using a computer system, it will be appreciated that a computer program for implementing at least part of the described methods and/or the described systems is envisaged as an aspect of the technology. The computer system may be any suitable apparatus, system or device, electronic, optical, or a combination thereof. For example, the computer system may be a programmable data processing apparatus, a computer, a Digital Signal Processor, an optical computer or a microprocessor. The computer program may be embodied as source code and undergo compilation for implementation on a computer, or may be embodied as object code, for example.

It is also conceivable that some or all functionality ascribed to the computer program or computer system may be implemented in hardware, for example by one or more application specific integrated circuits and/or optical elements. Suitably, the computer program can be stored on a carrier medium in computer usable form, which is also envisaged as an aspect of the technology. For example, the carrier medium may be solid-state memory, optical or magneto-optical memory such as a readable and/or writable disk for example a compact disk (CD) or a digital versatile disk (DVD), or magnetic memory such as disk or tape, and the computer system can utilize the program to configure it for operation. The computer program may also be supplied from a remote source embodied in a carrier medium such as an electronic signal, including a radio frequency carrier wave or an optical carrier wave.

It is accordingly intended that all matter contained in the above description or shown in the accompanying drawings be interpreted as illustrative rather than in a limiting sense. It is also to be understood that the following claims are intended to cover all generic and specific features of the technology as described herein, and all statements of the scope of the technology which, as a matter of language, might be said to fall there between.

Having described the technology, what is claimed as new and secured by Letters Patent is:

1. A method for coding an encounter between a patient and a healthcare practitioner, the method comprising:
capturing the encounter with a device associated with the healthcare practitioner;
transmitting the captured encounter over a network to a remote server for storage;
transmitting the captured encounter for review by a clinical documentation improvement coder (CDIC);
receiving input from the CDIC; wherein the input includes a patient encounter record which includes at least one code entry associated with the captured encounter; and, transmitting the patient encounter record over the network to a remote electronic medical record (EMR) system for storage.

2. The method according to claim 1 wherein the CDIC accesses the captured encounter in real-time.

3. The method according to claim 1 wherein the CDIC accesses the captured encounter subsequent to the captured encounter being stored on the server.

4. The method according to claim 1 wherein capturing the encounter includes capturing video and audio.

5. The method according to claim 1 wherein the CDIC includes artificial intelligence (AI).

6. The method according to claim 5 wherein the artificial intelligence employs voice recognition technology.

7. The method according to claim 1 further comprising:
transmitting the patient encounter record for receipt by the device associated with the healthcare practitioner;
receiving approval for the patient encounter record from the healthcare practitioner; and, storing the approved patient encounter record on the EMR system.

8. The method according to claim 7 wherein the patient encounter record is stored in the EMR system in accordance with health insurance portability and accountability Act (HIPAA) security requirements.

9. The method according to claim 1 further including: transmitting a secure text message from the device associated with the healthcare professional to the device associated with the CDIC.

10. The method according to claim 1 further including: transmitting the patient encounter record for receipt by an insurance provider.

11. The method according to claim 1 further including:
providing real-time access to the captured encounter to a remote healthcare provider and receiving advice related to the captured encounter from the remote healthcare provider.

12. The method according to claim 11 wherein the remote healthcare provider includes artificial intelligence (AI).

13. The method according to claim 12 wherein the artificial intelligence employs voice recognition technology.

14. The method according to claim 1 further including providing real-time access to the captured encounter to another CDIC and receiving advice related to coding from the another CDIC.

15. A system for coding an encounter between a patient and a healthcare practitioner, the system comprising:
a processor based device including a non-transitory computer readable medium that stores instructions which when executed cause the device to:
capture the encounter;
transmit the captured encounter over a network to a remote server for storage;
receive input from a remote clinical documentation improvement coder (CDIC); wherein the input includes a patient encounter record which includes at least one code entry associated with the captured encounter;
receive input regarding the patient encounter record from the healthcare practitioner; and, transmit the patient encounter record with the healthcare provider input over the network to a remote electronic medical record (EMR) system for storage.

16. The system according to claim 15 wherein the input from the healthcare practitioner includes approval of the patient encounter record.

17. The system according to claim 15 wherein the input from the healthcare practitioner includes at least one edit of the patient encounter record.

18. The system according to claim 15 wherein capturing the encounter includes capturing video and audio.

19. The system according to claim 15 wherein the CDIC includes artificial intelligence (AI).

20. The system according to claim 19 wherein the artificial intelligence employs voice recognition technology.

21. The system according to claim 15 wherein the executed instructions cause the device to further encrypt all transmissions for security.

22. The system according to claim 15 wherein the executed instructions cause the device to further transmit a secure text message for receipt by the CDIC.

23. The system according to claim 15 wherein the executed instructions cause the device to further transmit the patient encounter record for receipt by an insurance provider.

24. The system according to claim 15 wherein the executed instructions cause the device to further provide real-time access to the captured encounter to a remote healthcare provider and receive advice related to the captured encounter from the remote healthcare provider.

25. The system according to claim 24 wherein the remote healthcare provider includes artificial intelligence (AI).

26. The system according to claim 25 wherein the artificial intelligence employs voice recognition technology.

27. The method according to claim 1 further comprising prompting the healthcare practitioner to follow an encounter guide to streamline the patient encounter and ensure a plurality of steps are completed.
